(19) United States
(12) Patent Application Publication
(10) Pub. No.: US 2014/0051983 A1
(43) Pub. Date: Feb. 20, 2014

(54) ELECTROMAGNETIC INSTRUMENT TRACKING SYSTEM WITH METAL DISTORTION DETECTION AND UNLIMITED HEMISPHERE OPERATION

(71) Applicants: Tobias Schroeder, Melrose, MA (US); Daniel Eduardo Groszmann, Belmont, MA (US); John Whidden, Salt Lake City, UT (US); Steven Wayne Johnson, Salt Lake City, UT (US)

(72) Inventors: Tobias Schroeder, Melrose, MA (US); Daniel Eduardo Groszmann, Belmont, MA (US); John Whidden, Salt Lake City, UT (US); Steven Wayne Johnson, Salt Lake City, UT (US)

(21) Appl. No.: 13/586,663

(22) Filed: Aug. 15, 2012

(51) Int. Cl. A61B 5/05

(52) U.S. Cl. 600/424

(57) ABSTRACT

Electromagnetic tracking systems and methods for correcting hemispherical ambiguity are described. The system may include a single transmitter having three coils arranged in an industry-standard coil arrangement (ISCA). The tracking system may also contain a receiver having three coils arranged in an ISCA configuration, as well as a fourth coil having a different orientation then any of the other three coils of the receiver. The fourth coil may be used to determine the correct solution to the hemispherical ambiguity that can occur when tracking using two three-coil assemblies. Other embodiments are described.

### FIELD

This application relates generally to electromagnetic tracking systems. In particular, this application relates to electromagnetic tracking systems for use in image-guided surgery having unlimited hemisphere operation.

### BACKGROUND

Electromagnetic tracking systems have been used in various industries and applications to provide position and orientation information relating to objects. For example, electromagnetic tracking systems may be useful in aviation applications, motion sensing applications, retail applications, and medical applications. In medical applications, electromagnetic tracking systems have been used to provide an operator (e.g., a physician, surgeon, or other medical practitioner) with information to assist in the precise and rapid positioning of an instrument (such as a medical device, implant, tool, or other implement) in or near a patient's body during image-guided surgery. The electromagnetic tracking system provides positioning and orientation information for the instrument with respect to the patient's anatomy or to a reference coordinate system. The electromagnetic tracking system can also provide intraoperative tracking of the precise location of the instrument in relation to multidimensional images of a patient's anatomy. As the instrument is positioned with respect to the patient's anatomy, the displayed image is continuously updated to reflect the real-time position and orientation of the instrument being used.

Electromagnetic tracking systems may employ coils as receivers and transmitters. Typically, an electromagnetic tracking system is configured with an industry-standard coil architecture (ISCA). ISCA uses three collocated orthogonal quasi-dipole transmitter coils and three collocated orthogonal quasi-dipole receiver coils. The electromagnetic tracking systems typically contain three, three-axis coil assemblies that are generally used to derive the position and orientation of the instrument being used in the tracking system. One coil assembly is placed near the anatomy of interest to serve as a patient reference and a second coil assembly is located with the instrument. One of these coil assemblies acts as an electromagnetic transmitter and the other as an electromagnetic receiver. Alternatively, a third coil assembly may be placed at a fixed location within the surgical region of interest acting as an electromagnetic transmitter to transmit an electromagnetic field that is received by the first two coil assemblies acting as electromagnetic receivers. The combination of the image and the representation of the tracked instrument provide information that allows a medical practitioner to navigate the instrument to a desired location with an accurate position and orientation, as well as to display that location along with other reference structures or anatomy.

### SUMMARY

This application relates to electromagnetic tracking systems and methods for correcting hemispherical ambiguity. The system may include a single transmitter having three coils arranged in an industry-standard coil arrangement (ISCA). The transmitter may serve as patient reference, thus eliminating the need for an additional patient reference sensor. The tracking system may also contain an instrument receiver having three coils arranged in an ISCA configuration, as well as a fourth coil having a different orientation then any of the other three coils of the receiver. The fourth coil may be used to determine the correct solution to the hemispherical ambiguity that can occur when using two three-coil assemblies.

### BRIEF DESCRIPTION OF THE DRAWINGS

The following description can be better understood in light of the Figures, in which:

FIG. 1 illustrates a schematic of some embodiments of an exemplary electromagnetic surgical navigation system;

FIG. 2 shows a perspective view of some embodiments of an exemplary coil assembly;

FIGS. 3a-3b shows schematic representations of hemispherical ambiguity;

FIG. 4 shows a schematic of some embodiments of an exemplary electromagnetic tracking system;

FIG. 5 shows a schematic representation of some embodiments of an exemplary coil assembly;

FIG. 6 shows a schematic representation of some embodiments with respect to the interaction between exemplary coil assemblies in the electromagnetic tracking system; and

FIG. 7 shows a schematic of some embodiments of an exemplary electromagnetic tracking system.

The Figures illustrate specific aspects of the described systems and methods for electromagnetic tracking systems and methods for correcting hemispherical ambiguity. Together with the following description, the Figures demonstrate and explain the principles of the methods and structures produced through these methods. In the drawings, the thickness of layers and regions are exaggerated for clarity. The same reference numerals in different drawings represent the same element, and thus their descriptions will not be repeated. As the terms on, attached to, or coupled to are used herein, one object (e.g., a material, a layer, a substrate, etc.) can be on, attached to, or coupled to another object regardless of whether the one object is directly on, attached, or coupled to the other object or there are one or more intervening objects between the one object and the other object. Also, directions (e.g., above, below, top, bottom, side, up, down, under, over, upper, lower, horizontal, vertical, “x,” “y,” “z,” etc.), if provided, are relative and provided solely by way of example and for ease of illustration and discussion and not by way of limitation. In addition, where reference is made to a list of elements (e.g., elements a, b, c), such reference is intended to include any one of the listed elements by itself, any combination of less than all of the listed elements, and/or a combination of all of the listed elements.

### DETAILED DESCRIPTION

The following description supplies specific details in order to provide a thorough understanding. Nevertheless, the skilled artisan will understand that the described systems and methods for tracking position and orientation (P&O) of interchangeable instruments can be implemented and used without employing these specific details. Indeed, the described systems and methods for tracking P&O of interchangeable instruments can be placed into practice by modifying the described systems and methods and can be used in conjunction with any other apparatus and techniques conventionally used in the industry. For example, while the description below focuses on tracking P&O of instruments used in surgical navigation systems, the methods and systems for tracking P&O of instruments may be used in other systems in the fields of biomechanics, ergonomics, flight simulation and flight training, virtual reality applications, etc.

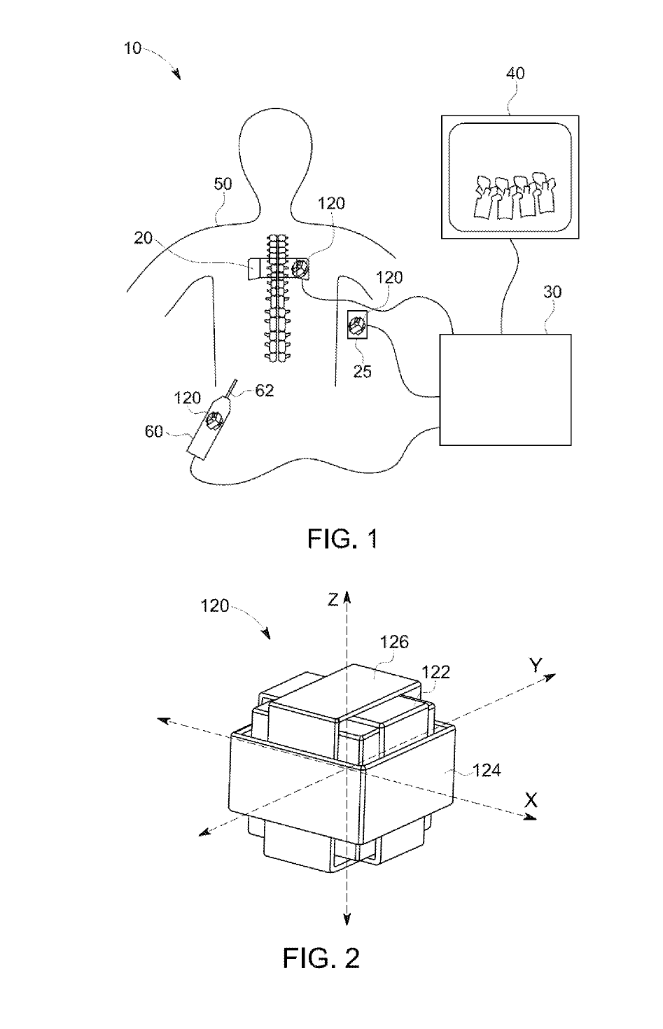

FIG. 1 illustrates some embodiments of a surgical tracking system 10 commonly used in the industry. In the tracking system 10 of FIG. 1, the tracking system includes industry-standard coil architecture (ISCA) coil packs (or assemblies) 120. In some configurations, the coil assemblies 120 may contain three approximately co-located, orthogonal quasi-dipole coils. The coil assemblies 120 may be used as transmitter coils (i.e., a transmitter coil trio) or as receiver coils (i.e., a receiver coil trio).

As shown in the embodiments depicted in FIG. 1, the tracking system 10 includes a patient reference receiver 20, a bedside transmitter 25, a processor 30, a monitor 40, and a receiver 60 connected to an instrument 62 used to affect the anatomy of a patient 50. The tracking system 10 may provide a visual representation of the anatomy of patient 50 displayed on the monitor 40 (or other display) and how the instrument 62 moves and interacts with the anatomy of the patient 50. Each of the receiver 20, the bedside transmitter 25, and the instrument receiver 60 may contain a coil assembly 120.

The coil assemblies 120 may contain a three-axis dipole coil transmitter or a three-axis dipole coil receiver. Each three-axis transmitter or receiver can be built so that the three coils exhibit the same effective area, are oriented orthogonally to one another, and are centered at the same point. FIG. 2 is an example of a dipole coil trio coil assembly 120 with a coil 122 oriented in the X direction, a coil 124 oriented in the Y direction, and a coil 126 oriented in the Z direction. The three coils can be spaced approximately equally about a center point, as shown in FIG. 2. If the coils are small enough compared to a distance between the transmitter and receiver, then the coil assembly may exhibit dipole behavior. The magnetic fields generated by the trio of transmitter coils may be detected by the trio of receiver coils. Using three approximately concentrically positioned transmitter coils and three approximately concentrically positioned receiver coils, for example, nine measurements may be obtained representing the interaction between each possible combination of receiver and transmitter coils. With the nine measurements analytical methods can solve for the six degrees of freedom that describe receiver P&O with respect to the transmitter coil trio. An example for such analytical methods is that described by Frederick H. Raab in Quasi-static Magnetic Field Technique for Determining Position and Orientation. IEEE Transactions on Geoscience and Remote Sensing. GE-19 (4): 235-243, October 1981.

FIG. 1 illustrates some relative positions of receiver 60 with a coil assembly 120, a patient reference receiver 20 with a coil assembly 120, and a bedside transmitter 25 with a coil assembly 120. In some embodiments, the coil assembly of receiver 60 may be secured to a medical instrument 62, the coil assembly of recevier 20 may be secured to a patient 50 (such as through a headset, band, or secured to a portion of the patient's anatomy such as through a bone screw, or the like), and the bedside transmitter 25 may be attached to an external reference point. In the preferred embodiment, the patient reference 20 is the transmitter and there is no bedside transmitter 25.

The mutual inductances between each of the three coils in the coil assembly of receiver 60 and each of the three coils in the coil assembly of the transmitter 20 can be measured. The position and orientation of the transmitter 20 with respect to the receiver 60 may then be calculated from the nine resulting mutual inductances of each of those coils and the knowledge of the coil characteristics. Thus, the position and orientation of the transmitter 20 with respect to the receiver 60 may be calculated by sensing the magnetic field generated by the transmitter 20. And with such a configuration, the position and orientation of the receiver with respect to the patient reference can be obtained even without using the bedside transmitter 25 coordinate frame.

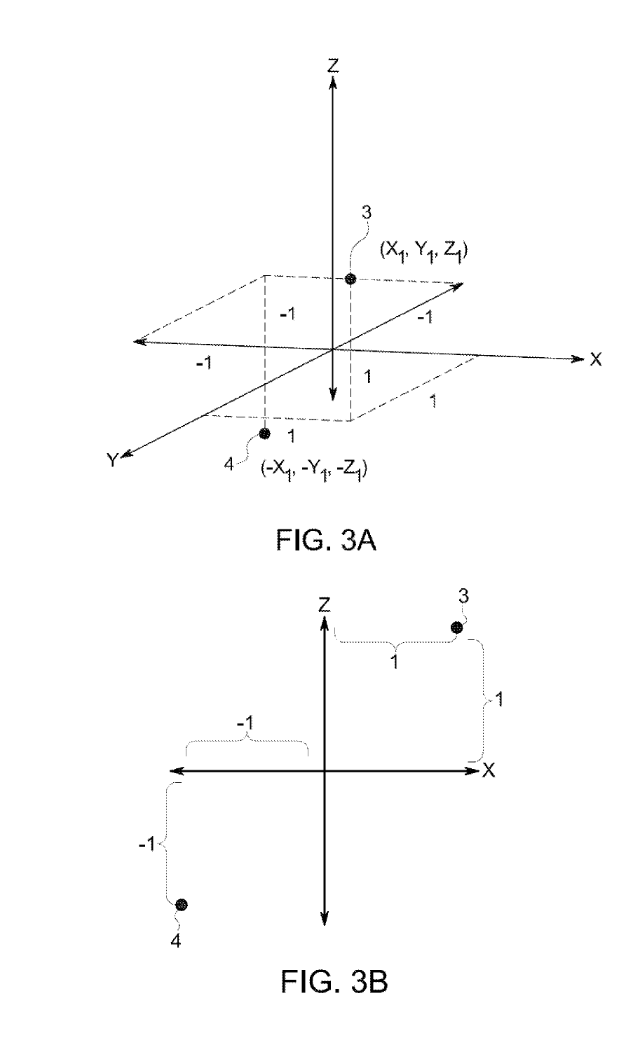

One limitation of tracking systems using the three coil assemblies 120 in the receiver 60 and a patient reference transmitter 20 is hemisphere ambiguity since tracking can take place at any position of the receiver around the transmitter. This is unlike the embodiment of using a bedside transmitter where tracking generally only takes place with the receiver in single hemisphere of the transmitter's reference frame. Hemisphere ambiguity arises when the receiver 60 is displaced 180 degrees about the origin as defined by transmitter 20, but has the same orientation. FIGS. 3a and 3b explain such an ambiguity.

FIGS. 3a and 3b show the relationship between a first point 3 with respect to a second point 4 that is located at a position that is diametrically opposite that of the first point. When the receiver 60 (with a coil assembly 120) is positioned at a first point 3 (x₁, y₁, z₁), and the transmitter 20 with a coil assembly 120 is positioned at the origin (per definition), the mutual inductances (or the magnetic field) measured between the receiver 60 at point 3 and the transmitter 20 are the same as when the receiver 60 is located at the second point 4 (−x₁, −y₁, −z₁) having the same orientation as at point 3. For example, when the first point 3 is at position (1 cm, 1 cm, 1 cm) and second point 4 is at position (−1 cm, −1 cm, −1 cm) with respect to the origin (0, 0, 0), the movement between the receiver 60 from point 3 to point 4 while keeping the transmitter 20 at the origin results in identical mutual inductances and magnetic fields being measured. This ambiguity in mutual inductances between transmitter and receivers results in the potential for the wrong coordinates to be calculated for the receiver with respect to the transmitter since the desired hemisphere is unknown.

In other embodiments, the hemisphere ambiguity may be eliminated by properly positioning two coil assemblies 120 on the receiver 60. In these embodiments, the coil assemblies 120 on the receiver 60 may be positioned a suitable distance apart so that they are distinguishable by the tracking system. If the receiver coil assemblies are positioned too close together, the tracking system may detect them as a single point as opposed to two separate points. Yet spacing the receiver coil assemblies a suitable distance apart required additional space and the medical instrument 62 may not be large enough to accommodate the two receiver coils positioned a suitable distance apart. So the use of additional receiver coil assemblies on the medical instrument 62 may be bulky, obtrusive, or otherwise awkward.

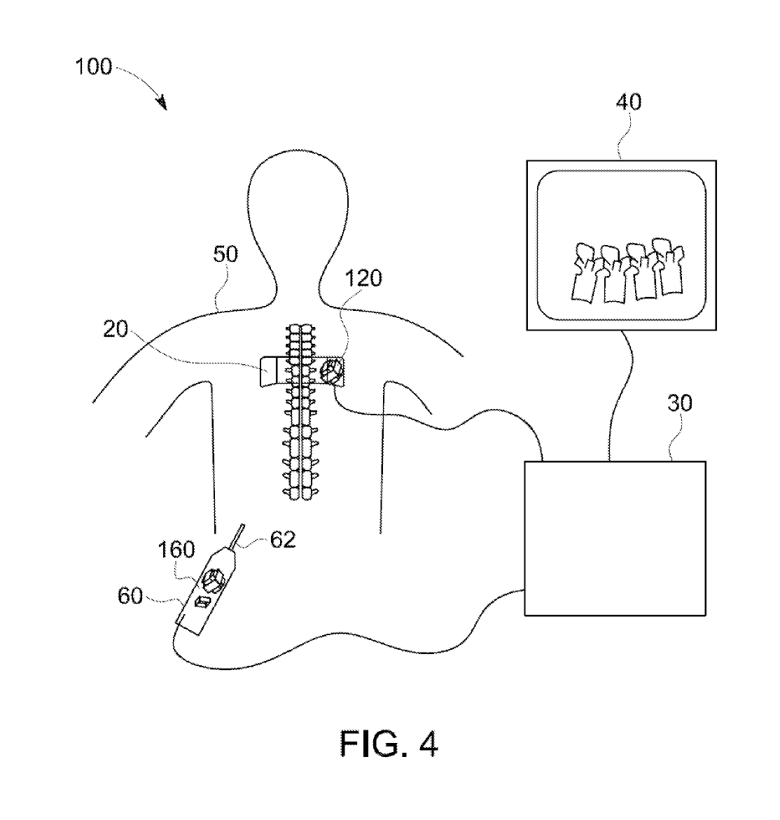

FIG. 4 illustrates embodiments of a tracking system which may be used to reduce or eliminate the hemisphere ambiguity. In FIG. 4, a surgical tracking system 100 including a transmitter 20, a processor 30, a monitor 40, and a receiver 60 with a four axis coil assembly 160 connected to an instrument 62 that is used to affect the anatomy of a patient 50. Similar to the tracking system 10, the tracking system 100 may provide a visual representation of the anatomy of a patient 50 that is displayed on monitor 40, as well as how display the instrument 62 moves and interacts with the anatomy of the patient 50.

The processor 30 may be any processor, computer, microcontroller, etc. configured to process information from the various other components and deliver display signals to the monitor 40. The processor 30 may perform any of the various processes discussed below with respect to determining and displaying the relative locations of the patient reference transmitter 20 and the receiver 60. The monitor 40 may be any monitor or display configured to display visual information related to the processes performed by the processor 30 and the tracking systems 100.

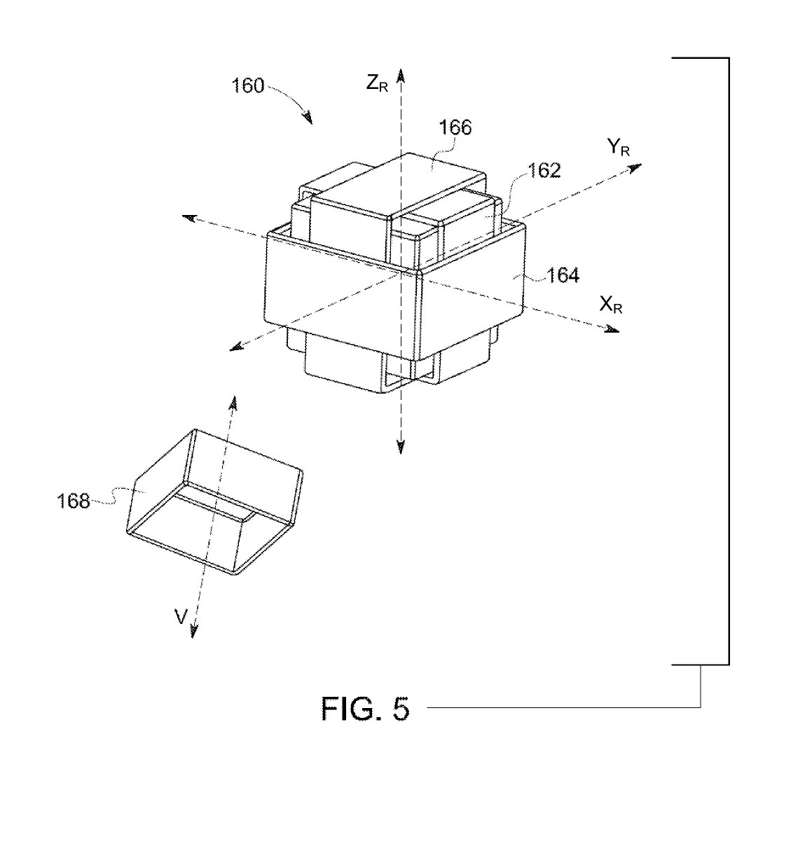

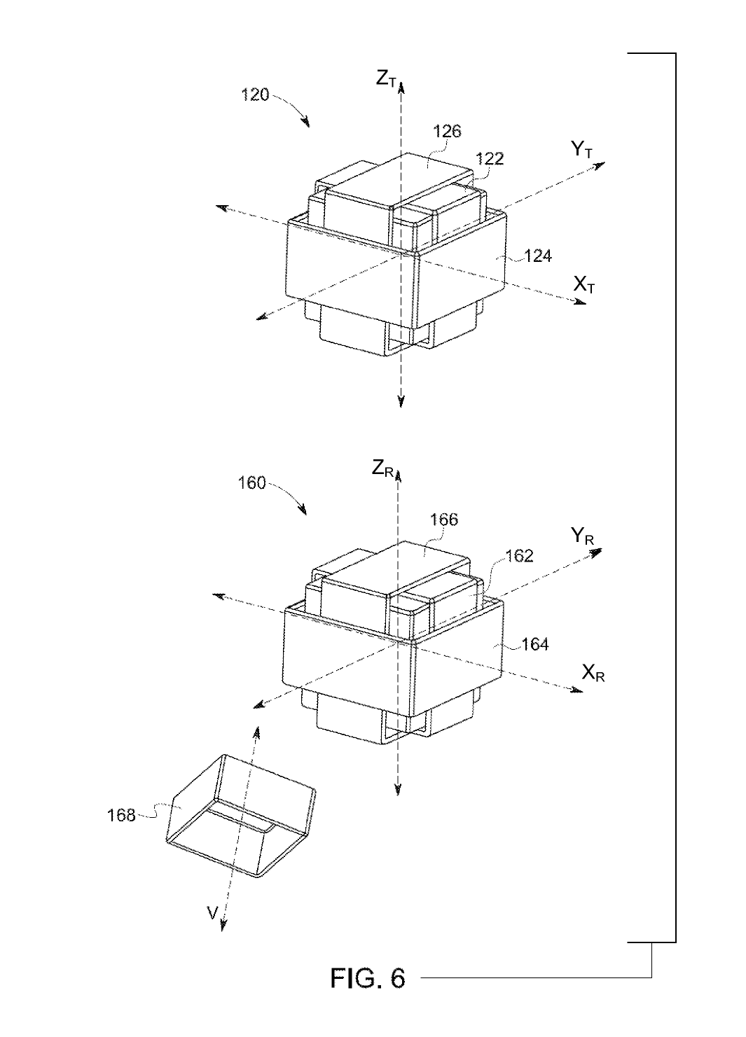

FIG. 5 illustrates the four axis coil assembly 160 used in the tracking system 100. The four axis coil assembly 160 may include a coil 162 oriented such that the electromagnetic field or magnetic axis is in the x_{R} direction, a coil 164 oriented in the y_{R} direction, a coil 166 oriented in the z_{R} direction, and a fourth coil 168 relative to a v axis. The fourth coil 168 may be positioned such that its associated v axis is not aligned with any of the x_{R}, y_{R}, or z_{R} axis. In some embodiments, the fourth coil 168 may be oriented such that the v axis is substantially not parallel with any of the x_{R}, y_{R}, and z_{R} axes. The position of the receiver 60 having the four axis coil assembly 160 may be calculated by measuring the mutual inductances between each of the four coils in the four axis coil assembly 160 in the receiver 60 related to the x_{R}, y_{R}, z_{R}, and v axis, and each of the three coils of the coil assembly 120 of transmitter 20 related to the x_{T}, y_{T}, and z_{T} axis.

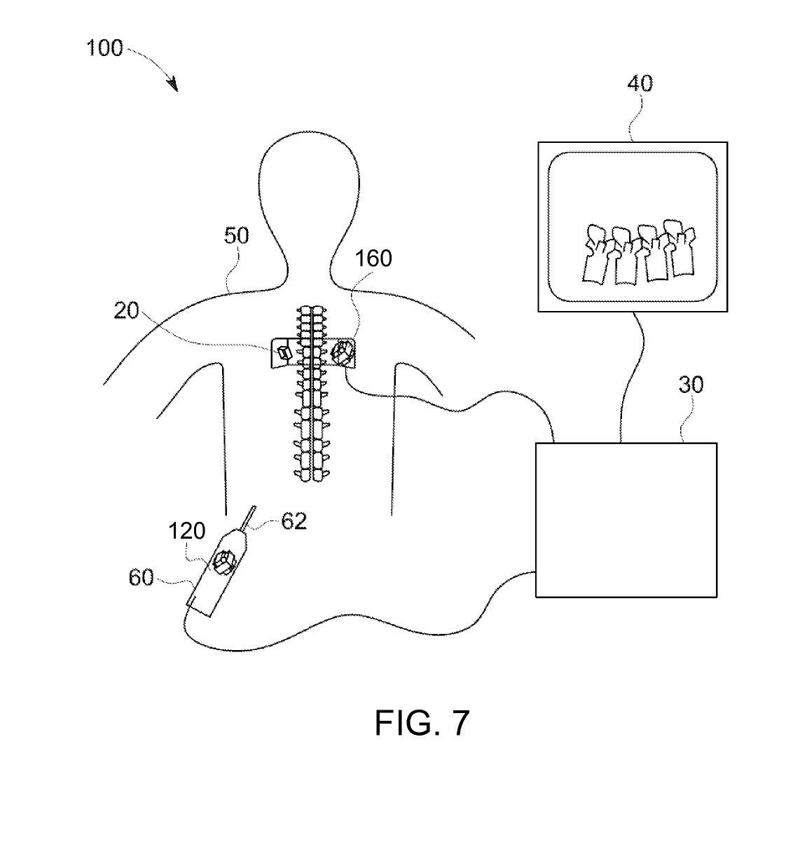

That is, as shown in FIG. 6, the mutual inductances between the coils associated with x_{T} axis and each of x_{R}, y_{R}, z_{R}, and v axis can be measured. Further, the mutual inductances between the coils associated with y_{T} axis and each of x_{R}, y_{R}, z_{R}, and v axis can be measured. And the mutual inductances between the coils associated with z_{T} axis and each of x_{R}, y_{R}, z_{R}, and v axis can be measured. Thus, a total of twelve mutual inductances can be measured between the four axis coil assembly 160 of the receiver 60 and the coil assembly 120 of the transmitter 20. The position and orientation of the four axis coil assembly 160, and consequently the receiver 60 and surgical instrument 62, with respect to the transmitter 20 may be calculated from the twelve resulting mutual inductances of each of those coils.

The fourth coil 168 can be used in methods for reducing or eliminating the hemisphere ambiguity when a pair of coil trio coil assemblies 120 is used in an electromagnetic tracking system. In some embodiments of these methods, the relative positions of the transmitter 20 with coil assembly 120 and receiver 60 with four axis coil assembly 160 can be determined. A first step of these methods may include calculating signals from the receiver coils 162, 164, and 166 related to x_{R}, y_{R}, and z_{R}, and each of the three coils of the coil assembly 120 of the transmitter 20 related to the x_{T}, y_{T}, and z_{T} axis. These signals may be processed with any analytical model to obtain solutions for the two possible receiver positions that may occur in opposite hemispheres.

Next, to identify which of the two positions is the correct one, a signal from the fourth coil 168 may be used. For the two possible positions, a magnetic field and a sensor coil model may provide the expected receiver voltage signals from each position. However, the position of the fourth coil 168 is different from that of the other three coils 162, 164, 166 of the receiver 60. When applying the two potential receiver positions, the corresponding positions and orientation of the fourth coil may be asymmetrical with respect to the coils 122, 124, 126 of the coil assembly 120 of the transmitter 20. As a result, the model-predicted signals from the fourth coil 168 may differ between the two hemispheres. The correct hemisphere may be identified as the one that provides the closest match between the measured and the model-predicted signal for the fourth coil 168. This type of hemisphere detection may function best when the fourth coil axis (v) is not parallel to any of the x_{R}, y_{R}, and z_{R} axes, as described above.

In some embodiments, an algorithm can be used as the analytical model for the hemisphere disambiguation. In these embodiments, the tracking system 100 may include the processes of solving ISCA signals for two potential receiver positions, using the model to calculate expected fourth coil signals for the two potential receiver positions, and then comparing the two expected signals against measured signal and choose the position that leads to the best agreement. In some embodiments, the algorithms may rely on measured signals and evaluate both a phase in receiver signal with respect to transmitter current, as well as an agreement between signals and their expected values based on an electromagnetic model.

By adding a fourth coil 168 to the receiver 60, the tracking system 100 may allow for tracking in any transmitter hemisphere without (or with limited) ambiguity. These embodiments may not only increase the available tracking volume (by double), but also allow for placing the transmitter at the center of an anatomical target region without the need of an additional coil assembly as a bedside transmitter. By having a single, anatomically centered transmitter 20, the transmitter 20 can be smaller with less power output since it is closer to the receiver 60. Additionally, the transmitter 20 can serve as patient anatomy reference point, eliminating the need for any additional patient reference receivers. As a result, the system architecture of the tracking system 100 may be simplified and tracking accuracy improved due to removing the patient reference receiver from the navigation chain. Accordingly, the tracking system 10 in FIG. 1 can be simplified. Thus, rather than comprising a bedside transmitter 25, a patient reference receiver 20, and an instrument receiver 60, the transmitter can be located at the center of the tracking space. Thus, the patient reference receiver can be removed and the transmitter can be moved from the bedside onto the patient, allowing the transmitter to operate both as a field generator and a patient reference.

Other embodiments may use other configurations of the electromagnetic coils. In some embodiments, the system may contain three large, non-dipole, non-collocated transmitter coils with three collocated quasi-dipole receiver coils. In other embodiments, the tracking system architecture may use an array of six or more transmitter coils spread out in space and one or more quasi-dipole receiver coils. In other embodiments, the tracking system architecture may use three approximately co-located, orthogonal quasi-dipole transmitter coils and one or more quasi-dipole magnetic sensor such as magneto-resistance, flux gate, or Hall-effect sensors. In yet other embodiments, a single quasi-dipole transmitter coil may be used with an array of six or more receivers spread out in space.

In some embodiments, the fourth coil may be positioned on either the receiver or transmitter. For example, as shown in FIG. 7, four axis coil assembly 160 may be positioned in the transmitter 20 and a coil assembly 120 in the receiver 60. In such embodiments, the solutions discussed above create valid solutions since the fourth coil provides the same additional data regardless of whether it is positioned in the transmitter or the receiver. Similarly, the receiver may be positioned on the patient and the transmitter may be positioned in the instrument. In other embodiments, the ISCA architecture may be modified such that the coils of each ISCA in the receiver and the transmitter are asymmetric, which may provide sufficient hemispherical differentiation to reduce or eliminate hemispherical ambiguity. In yet other configurations, the hemisphere disambiguation can work as well if the transmitter has a fourth coil and the receiver comprises a 3-coil assembly since the mutual inductance depends on the geometrical relationship among coils, not on whether they transmit or receive.

In addition to any previously indicated modification, numerous other variations and alternative arrangements may be devised by those skilled in the art without departing from the spirit and scope of this description, and appended claims are intended to cover such modifications and arrangements. Thus, while the information has been described above with particularity and detail in connection with what is presently deemed to be the most practical and preferred aspects, it will be apparent to those of ordinary skill in the art that numerous modifications, including, but not limited to, form, function, manner of operation and use may be made without departing from the principles and concepts set forth herein. Also, as used herein, the examples and embodiments, in all respects, are meant to be illustrative only and should not be construed to be limiting in any manner.

1. An electromagnetic tracking system, comprising:
a transmitter having three transmitter coils aligned such that each of the three transmitter coils is substantially perpendicular to each of the other transmitter coils;
a receiver having three receiver coils aligned such that each of the three receiver coils is substantially perpendicular to each of the other receiver coils, the receiver having a fourth coil oriented such that the fourth coil is substantially non-parallel with each of the three receiver coils; and
a processor configured to process signals from the transmitter and receiver to determine a relative position between the transmitter and the receiver.

2. The system of claim 1, wherein the each of the four receiver coils has a magnetic axis that is not parallel with the magnetic axes of the other receiver coils.

3. The system of claim 1, wherein the three receiver coils are substantially collocated and the fourth coil is not collocated.

4. The system of claim 1, wherein the transmitter is configured to be attached to an anatomy of interest of a patient.

5. The system of claim 4, wherein the receiver is configured to be coupled to a surgical instrument.

6. The system of claim 5, further comprising a monitor configured to display the anatomy of interest and the relative position of the surgical instrument to the anatomy of interest.

7. The system of claim 1, wherein the processor is further configured to use signals from the fourth coil to determine the actual location of the receiver with respect to the transmitter.

8. The system of claim 1, wherein the processor is further configured to resolve hemispherical ambiguity with a goodness of fit analysis.

9. A surgical navigation system, comprising:
a transmitter having three transmitter coils aligned such that each of the three transmitter coils is substantially perpendicular to each of the other transmitter coils;
a receiver having three receiver coils aligned such that each of the three receiver coils is substantially perpendicular to each of the other receiver coils, the receiver having a fourth coil oriented such that the fourth coil is substantially non-parallel with each of the three receiver coils;
a processor configured to process signals from the transmitter and receiver to determine a relative position between the transmitter and the receiver; and
a medical instrument attached to the transmitter or the receiver.

10. The system of claim 9, wherein the medical instrument is configured to be placed within the body of a patient.

11. The system of claim 9, wherein the each of the four receiver coils has a magnetic axis that is not parallel with the magnetic axes of the other receiver coils.

12. The system of claim 9, wherein the transmitter is configured to be connected to the body of the patient and the receiver is configured to be attached to the medical instrument.

13. The system of claim 12, further comprising a display operatively coupled to the processor and wherein the processor is configured to generate an image of the location of the medical instrument with respect to the body of the patient using the data relating to the location and orientation of the receiver.

14. The system of claim 9, wherein the receiver is configured to be connected to the body of the patient and the transmitter is configured to be attached to the medical instrument.

15. The system of claim 9, wherein each the four receiver coils has a different orientation than any other receiver coil.

16. The system of claim 9, wherein the processor is configured to evaluate the interaction between the receiver and the transmitter to resolve hemispherical ambiguity.

17. A surgical navigation system, comprising:
a tracking space configured to contain a patient;
a transmitter having three transmitter coils aligned such that each of the three transmitter coils is substantially perpendicular to each of the other transmitter coils, wherein the transmitter is located near the center of the tracking space and operates as both a field generator and a patient reference;
a receiver having three receiver coils aligned such that each of the three receiver coils is substantially perpendicular to each of the other receiver coils, the receiver having a fourth coil oriented such that the fourth coil is substantially non-parallel with each of the three receiver coils;
a processor configured to process signals from the transmitter and receiver to determine a relative position between the transmitter and the receiver; and
a medical instrument attached to the transmitter or the receiver.

18. The system of claim 17, wherein the system does not contain a patient reference receiver.

19. A method for correcting hemispherical ambiguity in a surgical navigation system, the method comprising:
providing a transmitter having three coils and a receiver having four coils;
calculating two possible solutions for the relative position and orientation of the receiver with respect to the transmitter based on signals from the three coils of the transmitter and three of the four coils of the receiver; and
identifying the correct solution based on signals from the fourth coil of the receiver.

20. The method of claim 19, wherein the each of the four receiver coils has a magnetic axis that is not parallel with the magnetic axes of the other receiver coils.

21. The method of claim 19, wherein the calculating is performed with a processor and wherein the receiver is configured to be coupled to a surgical instrument.

22. The method of claim 21, further comprising displaying a representation of the correct solution of the orientation and position of the receiver with respect to the transmitter.

23. The method of claim 21, wherein the transmitter is configured to be attached adjacent to an anatomy of interest inside of the body of a patient and the surgical instrument is configured to be used on the anatomy of interest.
